(19) United States
(12) Patent Application Publication
(10) Pub. No.: US 2025/0059571 A1
(43) Pub. Date: Feb. 20, 2025

(54) METHOD FOR CAPTURING PHYTOTOXINS IN A BIOLOGICAL REACTOR

(71) Applicant: CARBONWORKS, LIBOURNE (FR)

(72) Inventor: Philippe BOUDIER, CENON (FR)

(73) Assignee: CARBONWORKS, LIBOURNE (FR)

(21) Appl. No.: 18/721,295

(22) PCT Filed: Dec. 22, 2022
(86) PCT No.: PCT/EP2022/087404

§ 371 Date: Jun. 18, 2024

(30) Foreign Application Priority Data
Dec. 22, 2021 (FR) FR2114259

(51) Int. Cl. C12P 1/00; B01D 61/14

(52) U.S. Cl. CPC C12P 1/00; B01D 61/147; B01D 2311/2688

(57) ABSTRACT

A method for preparing a composition containing a biomass of microorganisms. This preparation method includes the recovery of phytotoxins from a fermentation juice obtained from culturing phytotoxin-producing microorganisms by membrane filtration of the biomass fermentation juice, with the phytotoxins being recovered in the retentate and the permeate being depleted in phytoxins. At least one addition cycle of the membrane of filtration is performed.

### FIELD

The present invention relates to a process for preparing a composition comprising a biomass of microorganisms, said preparation process comprising the recovery of phytotoxins from a fermentation juice obtained from the cultivation of microorganisms producing said phytotoxins.

### BACKGROUND

The processes for recovering biomasses and compounds such as phytotoxins included in these biomasses and/or the culture media are well known to those skilled in the art (WO2004/034790, Gijsbertsen-Abrahamse et al., Journal of Membrane science, 2006, 276 (1-2), pages 252-259, WO2020/058750, US2012/053344, Marcati et al., Algal Research 2014, 5, pages 258-263).

These processes generally consist of centrifugation of the culture medium or (micro) filtration, also allowing the purification of the biomass.

Microorganisms, and more particularly algae and microalgae, are today widely cultivated for their property of producing biomass rich in lipids, proteins, carbohydrates, etc.

In parallel with the multiplication of cells, microorganisms secrete molecules of interest including phytotoxins.

These phytotoxins, present mainly in the culture medium, and more precisely in the fermentation juice, are generally eliminated during the recovery and purification of the biomass.

However, phytotoxins secreted by microorganisms have interesting biological properties depending on their nature, and can be used in various fields such as the agricultural or pharmaceutical industry (WO2004/052097 and US2007/166266).

The isolated phytotoxins can also be used to adjust the concentrations of the batches of biomass produced.

Indeed, the quality and concentration of compounds, including phytotoxins, present in biomass vary depending on the cultivation conditions.

Finally, in the context of an environmental issue, it is interesting to be able to recover phytotoxins, in particular phytotoxins harmful to the environment, and thus limit their propagation in nature.

The inventors have thus developed a process for recovering phytotoxins from a fermentation juice obtained from the culture of microorganisms.

### SUMMARY

The present invention relates to a process for preparing a composition comprising a biomass of microorganisms, said preparation process comprising the addition of phytotoxins as an additive to said biomass, characterized in that the phytotoxins are obtained by a process comprising the steps of:
a. membrane filtration of a biomass fermentation juice so as to retain the phytotoxins in the retentate, said biomass being obtained from a culture of phytotoxin-producing microorganisms,
b. recovery of a retentate comprising said phytotoxins, and
c. recovery of a first permeate depleted in phytotoxins, said permeate having a phytotoxin content lower than that of the fermentation juice,
   followed if applicable by at least one additional cycle of membrane filtration of the retentate obtained in step (c) of the previous cycle with the recovery of at least a second retentate comprising said phytotoxins and at least a second permeate depleted in phytotoxins having a phytotoxin content lower than that of the first permeate.

Obtaining the phytotoxins according to the invention can thus comprise m additional cycles of membrane filtration, and the recovery of m+1 retentates comprising the phytotoxins and m permeates depleted in phytotoxins, m being an integer equal to or greater than 1.

The different membrane filtration cycles can be carried out on the same filtration membranes or on different membranes.

The same goes for filtration cycles, each of which may include similar or different membranes.

In the context of the present invention, the final concentration of phytotoxins obtained in the permeate recovered after the last filtration cycle can be determined by the equation:

$${C}_{k [ λ ]} = {C}_{[ λ ]} * \frac{X}{X - 1} ( 1 - {X}^{R - 1} ) * {X}^{R}$$

in which I is the number of cycles m+1, concentration of the membrane and R is the molecular rejection rate of the membrane.

Phytotoxins are molecules with a molecular weight between 300 and 3500 Da.

Finally, the invention concerns the use of the permeate resulting from the process as food in a microorganism cultivation process.

### BRIEF DESCRIPTION OF THE FIGURES

FIG. 1 represents an embodiment of the invention.

FIG. 2 represents the number of filtration cycles n as a function of the concentration of phytotoxins (cylindrospermopsin) in the retentate.

FIG. 3 represents the evolution of the concentration of phytotoxins (cylindrospermopsin) in the retentate as a function of the number of membrane filtration cycles.

FIG. 4 represents the yield of phytotoxins recovered at each membrane filtration step as a function of membrane type.

FIG. 5 represents the evolution of filtration on a 1000 Dalton filter of the maitotoxin toxin (C164H258O68S2) with a size of 3380 Daltons by LC-MS/MS.

### DETAILED DESCRIPTION

The present invention relates to a process for preparing a composition comprising a biomass of microorganisms, said preparation process comprising the addition of phytotoxins as an additive to said biomass, characterized in that the phytotoxins are obtained by a process comprising the steps of:
a. membrane filtration of a biomass fermentation juice so as to retain the phytotoxins in the retentate, said biomass being obtained from a culture of phytotoxin-producing microorganisms,
b. recovery of a retentate comprising said phytotoxins, and
c. recovery of a first permeate depleted in phytotoxins, said permeate having a phytotoxin content lower than that of the fermentation juice,
   followed if applicable by at least one additional cycle of membrane filtration of the retentate obtained in step (c) of the previous cycle, with the recovery of at least a second retentate comprising said phytotoxins and at least a second permeate depleted in phytotoxins having a phytotoxin content lower than that of the first permeate.

In the context of the present invention, phytotoxins are understood to mean molecules or chemical substances secreted by microorganisms during their cultivation, that is to say in parallel with the formation of a biomass of said microorganisms.

The term “phytotoxin” thus brings together biotoxins produced by plants, fungi (also called mycotoxins), and algae (also called phycotoxins). In the context of the present invention, the phytotoxins are advantageously phycotoxins. Examples of phycotoxins are domoic acid, okadaic acid, saxitoxins, brevetoxins and even ciguatoxins.

In the context of the present invention, the term “biomass” means a set of microorganism cells (i.e. several thousand cells and more) produced by cultivating said microorganisms on a culture medium and separated from this culture medium at the end of cultivation after harvest. As a result of their culture conditions, the microorganism cells in the biomass all have substantially the same composition. The biomass of microorganisms is a composition which includes cells of microorganisms cultivated with water and possibly traces of nutrients and other elements present in the culture medium.

In the context of this application, the biomass is microalgae biomass. The microalgae biomass can be raw biomass, lysed biomass, homogenized biomass or dried biomass. In the context of this application, the term “raw biomass” designates biomass obtained after harvest, without other modification such as cell lysis or homogenization. The raw biomass comprises at least 70% water, and up to 90% water, preferably 80 to 85% water. Raw biomass also includes raw biomass washed after harvest in order to eliminate certain nutrients and other elements present in the growing medium.

A “lysed biomass”, in the context of the present application, means a biomass of microalgae in which at least 50% of the cells are lysed, preferably at least 70%, more preferably at least 80%, 85%, 90%, 95%, up to 100% of cells are lysed. For this purpose, the lysis is carried out by a human according to known methods, such as mechanical, chemical or enzymatic lysis, well known to those skilled in the art (WO2015/095688, WO201 1/153246, U.S. Pat. No. 6,750,048 and WO201 5/095694, WO2018/178334, WO2020/144330, WO2020/053375, CN106749633, CN102433015, CN1 117973, Michelon et al. 2016). Raw biomass may include lysed cells due to the harvesting step. However, the ratio of lysed cells in the raw biomass is generally very low, less than 50%, with harvesting carried out to avoid significant biomass damage. With the exception of materials added for biomass lysis, lysed biomass contains only algal biomass in a transformed state.

“Dry biomass” in the context of this application means that the biomass comprises less than 10% water, preferably 1 to 10% water, more preferably 2 to 7%. Dry biomass is biomass obtained after a drying step carried out under conditions defined by a human being or a computer under the control of a human being.

In the context of the present application, the term “microalgae culture” designates an industrial cultivation method controlled by humans and comprising the seeding of a culture medium chosen by said human with cells of said microalgae, allowing the cells to multiply and grow to a certain high cell density in the culture medium. For the avoidance of doubt, “microalgae cultivation” according to the invention does not mean the harvesting of microalgae found in nature.

In one embodiment, the microalgae biomass may be raw biomass, lysed biomass, homogenized biomass or dried biomass.

“Microalgae cultures” includes a step a) of cell growth and accumulation of metabolites, and a step b) of harvesting the biomass by separating all the microalgae cells forming the culture medium. Cell growth and accumulation may be a single stage where growth conditions allow the accumulation of metabolites. In another embodiment, the growth of the cells and the accumulation can be carried out in two successive steps, where step a1) allows the growth of the cells at a certain density, and is followed by step a2) of maturation where metabolites are accumulated in cells without appreciably affecting cell density in the culture medium. In certain embodiments, the growth step a1) also allows a certain level of accumulation of metabolites reinforced by the maturation step a2). In certain embodiments, maturation step a2) also allows a certain level of improvement in cell density.

According to the invention, phytotoxins are metabolites.

The term “culture medium” in the context of the present application designates an aqueous composition comprising the nutrients necessary for the growth of microalgae. The culture medium comprises a source of nitrogen, a source of phosphorus, salts, and/or vitamins, and/or trace elements and other nutrients well known to those skilled in the art. For heterotrophic or mixotrophic cultures, the culture medium also includes a source of organic carbon metabolized by the microalgae, while for autotrophic cultures, a source of mineral carbon such as CO2 is present in the culture medium.

The carbon source can be a source of organic carbon for a culture in heterotrophic or mixotrophic mode. An “organic carbon source” in the context of the present application means an aliphatic molecule, in particular a polyol, an organic acid or a carbohydrate. Examples of such complex carbon sources are glycerol, glucose, fructose, xylose, sucrose, cellulose derivatives, lactic acid and salts, starch, starch derivatives, and their mixtures, and any complex product comprising at least one of these molecules.

The carbon source can be carbon dioxide for growing in autotrophic mode where light provides energy to cells to incorporate carbon during photosynthesis. This carbon dioxide is generally supplied to cells throughout the culture in a gaseous or pre-dissolved form.

The methods for cultivating microorganisms are well known to those skilled in the art, whether in heterotrophic, autotrophic or mixotrophic mode.

The microorganisms according to the invention are cultivated in biological reactors, reactors within which biological phenomena develop, such as growth of pure cultures or microorganisms or of a consortium of microorganisms.

Phytotoxins produced by microorganisms are mainly found in the fermentation juice obtained during cell culture. The term “fermentation” thus designates the operation which makes it possible to produce biomass and/or bioconversion products by cultivating microorganisms.

In the context of the invention, the term “fermentation juice”, also called culture broth, refers to the liquid phase included in the biological reactor. The fermentation juice thus includes, among other things, water, nutrients and compounds secreted and/or produced by microorganisms.

The fermentation juice and the biomass are separated using the usual separation methods. We will particularly mention centrifugation (plate centrifuge or sedicator), and filtration (plate filter, filter press, ceramic or organic tangential filtration). These techniques are well known to those skilled in the art who will know determine, depending on the nature and characteristics of the biomass and the fermentation juice, the most appropriate method.

In one embodiment, the process for recovering phytotoxins from a fermentation juice obtained from the cultivation of microorganisms producing said phytotoxins is thus preceded by a step of separating the biomass and the fermentation juice.

The fermentation juice can also be obtained from a fermentation must obtained by a process of fermentation of said microorganisms in a culture medium comprising an aqueous culture medium and nutrients necessary for the growth of said microorganisms.

By “fermentation must” we mean the liquid/solid mixture produced by the microorganisms. In this case, the fermentation must is separated from the biomass of microorganisms according to methods well known to those skilled in the art, before extracting the fermentation juice.

Once the fermentation juice is separated, the phytotoxins are extracted according to the process defined in this application. The process for recovering phytotoxins from the fermentation juice of the biomass produced comprises at least one membrane filtration cycle, this filtration cycle comprising the following steps:
(a) membrane filtration of the fermentation juice of the biomass so as to retain the phytotoxins in the retentate,
(b) recovery of the retentate comprising said phytotoxins, and
(c) recovery of the permeate depleted in phytotoxins, said permeate having a phytotoxin content lower than that of the fermentation juice.

By “retentate” we mean all the molecules and/or particles which are retained during the membrane filtration process. On the contrary, by “permeate” or “filtrate” we mean the molecules and/or particles which have passed through the membrane. In the context of the invention, the phytotoxins are mainly recovered in the retentate.

The filtration cycle may include one or more additional cycle(s) of membrane filtration of the retentate obtained in step (c).

In one embodiment, the process comprises at least one membrane filtration cycle of the retentate obtained in step (c), then leading to the recovery of at least a second retentate comprising said phytotoxins and a second permeate depleted in phytotoxins having a phytotoxin content lower than that of the first permeate.

In another embodiment, the method according to the invention comprises more than one additional membrane filtration cycle. Generally speaking, the process may comprise m additional cycles of membrane filtration, m being an integer between 1 and 10. In one embodiment, m is between 1 and 10, more particularly m is chosen from the values of 1, 2, 3, 4, 5, 6, 7, 8, 9 and 10. In a preferred embodiment, m has the value 1, 2 or 3. In another preferred embodiment, m is 1 or 2.

The process according to the invention comprising m additional membrane filtration cycles leads to the recovery of m+1 retentates comprising phytotoxins and m permeates depleted in phytotoxins.

Membrane filtration cycles are carried out with a single membrane or on a new similar or different membrane. By “identical or different membrane” is meant membranes whose intrinsic characteristics are identical or different.

In one embodiment, each filtration cycle of the process according to the invention is carried out on the same filtration membrane. The permeate obtained from the membrane filtration is thus filtered again with this first membrane in the additional filtration cycle.

In another embodiment, each filtration cycle is carried out on a new membrane, it being understood that this may have intrinsic characteristics similar or different from the membrane of the previous filtration cycle.

The number of additional membrane filtration cycles m or the number of total membrane filtration cycles n are determined based on several parameters, including the phytotoxin content of the fermentation juice, the concentration factor of the filtration membrane and the desired final concentration of phytotoxins in the permeate recovered after the last filtration cycle.

Membranes are characterized by their intrinsic properties including pore size, cut-off threshold, selectivity and permeability. A person skilled in the art will be able to determine the type of membrane to use according to the process parameters, such as the number of filtration stages, the concentration factor, the concentration of phytotoxins desired in the final permeate, etc.

In the context of the present invention, the membranes are membranes whose cut-off threshold is lower than the molar mass of the filtered phytotoxins. A person skilled in the art will be able to determine the value of the cut-off thresholds, and therefore the appropriate membranes to use.

The cutoff threshold can range from approximately 100 to 1500 Da, in particular from approximately 100 Da to approximately 1000 Da, or even from approximately 100 Da to approximately 700 Da, in particular 100 Da, 200 Da, 300 Da, 400 Da, 500 Da or 600 Da.

In one embodiment, the membranes thus have pores whose diameter is between 0.6 and 0.7 nm. In parallel, the molecular weight cutoff threshold of the membranes is between approximately 100 and 300 Da. Particular examples of membranes are the membranes used in reverse osmosis. Such membranes are the SEPA CF II membranes marketed by Ge Osmonics and SW C4 marketed by Hydranautics.

The final concentration of phytotoxins in the permeate recovered after the last membrane filtration cycle, as a function of the number of cycles m+1, can be determined by the following equation:

$${C}_{k [ λ ]} = {C}_{[ λ ]} * \frac{X}{X - 1} ( 1 - {X}^{R - 1} ) * {X}^{R}$$

in which λ is the number of cycles m+1, X is the membrane concentration factor and R is the membrane molecular rejection rate, and the values of X and R are known.

It is also possible to determine the overall efficiency nₖ of a number of passages n in a membrane according to the following formula:

$${η}_{k} = {( \frac{1}{( 1 - R ) * ( {X}^{\frac{1}{n}} - 1 )} )}^{n}$$

In the case where a single filtration cycle is used, the efficiency nₖ is calculated according to the formula:

$${η}_{k} = \frac{1}{( 1 - R ) * ( X - 1 )}$$

Advantageously, the recovered permeate has a phytotoxin content lower than that of the fermentation juice. In one embodiment, the permeate has a phytotoxin content of less than 10% by weight relative to the total weight of the biomass, or even less than 8%, or even less than 7%, preferably less than 6%. In another embodiment, the phytotoxin content is between 0 and 5% by weight relative to the total weight of the biomass. Preferably, the phytotoxin content is between 0 and 4%, preferably between 0 and 3%, and more preferably between 0 and 2%. In another preferred embodiment, the phytotoxin content is less than 1% by weight relative to the total weight of the biomass. In the context of the present invention, the limit of 0% by weight relative to the total weight of the biomass corresponds to the detection limit of phytotoxins present in the permeate.

The m+1 retentates comprising the phytotoxins recovered from the first membrane filtration cycle and the retentates from the m additional membrane filtration cycles are recovered and possibly combined.

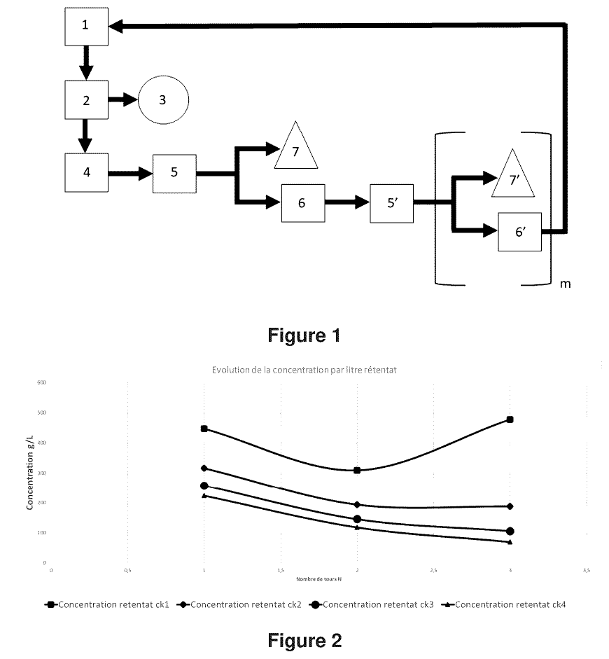

An embodiment of the process for recovering phytotoxins from a fermentation juice obtained from microorganisms is shown in FIG. 1. After the microorganisms have been cultivated in the biological reactor (1), the fermentation juice (2) and the biomass (3) are separated. The biomass (3) is recovered, possibly treated and/or transformed depending on its fate. In parallel, the fermentation juice (4) is purified by membrane filtration (5). The retentate (7) is isolated, while the permeate (6) can be isolated or undergo a new filtration cycle of membrane filtration m, leading to obtaining the retentate m+1 and the permeate m. The permeate thus obtained is reintroduced into the biological reactor (1).

Microorganisms and cultured microorganisms are well known to those skilled in the art. These include bacteria, yeasts, and even protists, and more particularly algae and microalgae. According to a preferred embodiment, the cultured microorganisms are protists. By protist we mean all unicellular eukaryotic microorganisms.

Microalgae (Chlorophytes such as Chlorella, Senedesmus, Tetraselmis, Haematococcus; Charophytes, chrysophytes including diatoms; Nannochloropsis; Euglenophytes such as Euglena, Phacus; Rodophytes including Galdieria, etc.), unicellular fungi (Thrautochytrids such as Schizochytrium, Aurantiochytrium, etc.), cyanobacteria (Anabaena, Nostoc, Microcistis, Arthrospira, Spirulina, etc.) or heterotrophic flagellates (Crypthecodinium etc.) are part of the protists group.

In one embodiment, the microorganisms according to the invention are algae and microalgae belonging preferentially to the classes of Chiorophytes, Rodophytes, Thraustochytrids, Diatoms and Dinoflagellates.

When the microalgae are diatoms, they can be chosen from the following genera: Nitzschia, Navicula, Gyrosigma, Phaeodactylum, Thalassiosira, etc.

When the algae or microalgae are Dinoflagellates, they may be chosen from the species: Alexandrium, Fragilidinium, Coolia, Ostreopsis, Fukuyoa, Gambierdiscus, Goniodoma, Pyrocystis, Pyrodinium, Pyrophacus, Ceratium, Tripos, Lingulodinium, Amylax, Gonyaulax verior Gonyaulax, Ceratocorys, Protoceratium, Amphisolenia, Dinophysis, Histioneis Ornithocercus, Phalachroma, Triposolenia, Sinophysis, Pseudophalacroma, Ansanella, Asulcocephalium, Baldinia, Biecheleria, Biecheleriopsis, Borghiella, Cystodinium, Leiocephalium, Pelagodinium, Phytodinium, Piscinoodinium, Polarella, Symbiodinium, Woloszynskia, Amyloodinium, Cryptoperidiniopsis, Paulsenella, Pfiesteria, Pernambugia, Dubosquodinium, Naiadinium, Scrippsiella, Theleodinium Apocalathium Crypthecodinium Stoeckeria, Chimonodinium, Thoracosphaera, Aduncodinium glandula, Tintinnophagus acutus, Azadinium, Amphidoma, Azadinium dexteroporum, Azadinium polongum, Azadinium concinnum, nium caudatum, Durinskia, Galeidinium, Kryptoperidinium, Unruhdinium, Blixaea, Ensiculifera, Pentapharsodinium, Monovela clade: Amphidiniopsis, Archaeperidinium, Herdmania, Islandinium, Protoperidinium americanum, P. fusiforme, P. fukuyoi, P. monovelum, P. parthenopes, Peridinium clade: Peridinium stricto: Protoperidinium abei, P. bipes, P. conicum, P. crassipes, P. divergens, P. denticulatum, P. elegans, P. excentricum, P. leonis, P. pallidum, P. pellucidum, P. pentagonum, P. punctulatum, P. thorianum, P. thulesense, Kolkwitziella, Diplopsalioideae III and Oceanica clade: Diplopsalopsis, Niea, Qia, Gotoius, Protoperidinium claudicans, Protoperidinium depressum, Diplopsalis caspica, D. lenticula, Preperidinium meunier/′, Heterocapsa, Blepharocysta, Podolampas, Roscoffia, Prorocentrum dentatum, P. donghaiense, P. emarginatum, P. fukuyoi, P. mexicanum, P. micans, P. minimum, P. rhathymum, P. shikoense, P. texanum, P. triestinum, P. tsawwassenense, Prorocentrum belizeanum, P. bimaculatum, P. concavum, P. consutum, P. foraminosum, P. maculosum, P. levis, P. lima, Prorocentrum glenanicum, P. panamense, P. pseudopanamense, Plagiodinium, Prorocentrum cassubicum, Akashiwo, Chytriodinium, Dissodinium, Erythropsidinium, Gymnodinium, Gymnoxanthella, Gyrodiniellum, Lepidodinium, Nematodinium, Nusuttodinium, Paragymnodinium, Pellucidodinium, Pheopolykrikos, Polykrikos, Proterythropsis, Spiniferodinium, Warnowia, Brachidinium, Karenia, Karlodinium, Takayama, Gyrodinium, Amphidinium, Amphidinium mootonorum, A. herdmanii, A., A. carterae, Torodinium, Kapelodinium, Esoptrodinium, Jadwigia, Tovellia, Blastodinium navicula, B. mangini, B. galatheanum, Blastodinium spinulosum, B. crassum, B. pruvoti, B. inornatum, Blastodinium contortum, Blastodinium oviforme, Ptychodiscus noctiluca, Ailadinium, Amphidiniella, Bysmatrum, Glenoaulax, Glenodiniopsis, Gloeodinium, Hemidinium, Heterodinium, Madanidinium, Oodinium, Palatinus, Parvodinium, Peridinium sociale, Peridiniopsis borgei, Pileidinium, Pseudadenoides, Rufusiella, Sabulodinium, Stylodinium, Thecadinium, Zooxanthella, Ankistrodinium, Apicoporus, spinodinium, Balechina, Ceratoperidinium, Margalefidinium, Cucumeridinium, Levanderina, Moestrupia, Testudodinium, and Togula.

According to a particular embodiment of the invention, the protists are chosen from the genera Chlorella, Galdieria, Euglena, cyanobacteria, diatoms and dinoflagellates including in particular the genus Gambierdiscus.

The phytotoxins secreted by microorganisms are small molecules well known to those skilled in the art.

In the context of the present invention, phytotoxins are molecules secreted by cultivated microorganisms and whose molecular weight is between 300 and 3500 Da, in particular between 300 and 3000 Da. In another embodiment, the phytotoxins according to the invention have a molecular weight of between 500 and 3500 Da, between 300 and 2900 Da, or even between 500 and 2800 Da, or even between 500 and 2500 Da.

Examples of phytotoxins, but not limited to, are compounds belonging to the families of microcystins, brevetoxins, saxitoxins, anatoxins a, okadaic acids, ciguatoxins, amphidinols, cylindrospermopsins, cyclic imines, such as gymnodimines, spirolides, pinatoxins, pteraitoxins, prorocentrolides and spiroprorocentrolides, ovatoxins, palytoxins and anabaenopeptins. Among the phytotoxins, mention may in particular be made of kanoic acid, ciguatera, karlotoxin, palytoxin, tetrodotoxin, yessotoxin, gambierol, amphidinolide, dinophysis, ostreocins, gonyautoxins, maitotoxin or scaritoxin.

The invention also relates to the use of phytotoxins obtained according to the process of the invention. The phytotoxins present in the retentates can be purified by methods known to those skilled in the art. The phytotoxins thus isolated and purified can be used in the agricultural industry, as pesticides, or in the pharmaceutical or cosmetic industry as biologically active compounds. Another application of phytotoxins, or even retentates rich in phytotoxins, is their use as an additive in a biomass of microorganisms. Indeed, the composition of the harvested biomasses varies depending on the cultivation conditions, and the addition of phytotoxins is sometimes necessary to obtain standardization of the composition of batches of biomass. According to the invention, “standardization” means the reproduction of the characteristics of the biomass according to predetermined standard values, and more particularly of the composition of the biomass, in relation to a standard or defined characteristics. The predefined standards or characteristics may be standard values determined by those skilled in the art based on a particular application of the standardized biomass obtained. It may also involve meeting official standards or specifications, such as for example the EC 1107/2009 regulation list on the use of biopesticides of the European Union or the Ecophyto II+ specifications of the ANSES (National Agency for Food, Environmental and Occupational Health Safety) in France, or any other official regulation relating to the use of phytotoxins. Standardization also allows industrialization and control of the industrial products thus obtained with a view to their marketing.

The invention also relates to the use of the permeate(s) obtained according to the process of the invention. At the end of the process, the permeates comprising small quantities of phytotoxins are used as a culture medium, particularly in a biological reactor. It is understood that depending on the composition of these retentates, other elements can be added in order to obtain a culture medium suitable for microorganisms. In one embodiment, the nth permeate obtained after n membrane filtration cycles is reintroduced into the biological reactor. This closed system thus makes it possible to recycle the permeates obtained. In another embodiment, the permeates are isolated, possibly combined, before being used as a culture medium. These permeates can also be stored for later uses.

### EXAMPLES

### Example 1

The objective is to recycle the permeate still containing active molecules, the permeate will be used as feed for a number of loops n until the total extraction of the mass concentration of the initial feed.

Consequently the result of the concentration after a first turn of the membrane will depend on the concentration of the permeate, hence C_{k[λ]} the concentration of the feed after lambda turn.

$${C}_{k [ λ ]} = {C}_{P [ ( λ - 1 ) ]} * {X}^{R} Avec λ \geq 1 et Si$$

$$λ = 1$$

$${C}_{k} = {C}_{0}$$

$$where$$

$${C}_{P [ λ ]} = {C}_{[ λ ]} * \frac{X}{X - 1} ( 1 - {X}^{R - 1} )$$

$$and$$

$${C}_{k [ λ ]} = {C}_{[ λ ]} * \frac{X}{X - 1} ( 1 - {X}^{R - 1} ) * {X}^{R}$$

To obtain the most concentrated batches possible between each turn in the membranes, the specificities of each phytotoxin must be taken into account.

In the case of cylindrospermopsin, a membrane size of less than 415 Da and with a high concentration factor should be favored.

It is interesting to have membranes with different concentration factors in order to minimize the number of turns in the membrane but also to recover the most concentrated retentate, also ensuring optimization of the yield at each turn n in the membrane.

FIG. 2 represents the number of filtration turns n as a function of the concentration of phytotoxins (cylindrospermopsin) in the retentate. The concentration factors denoted ck have the following values: ck1=5, ck2=2.5, ck3=1.6 and ck4=1.25.

When the concentration factor is greater than or equal to 5 (ck1), each batch of retentates is more concentrated after each passage through the membranes, thus ensuring much more varied batch possibilities.

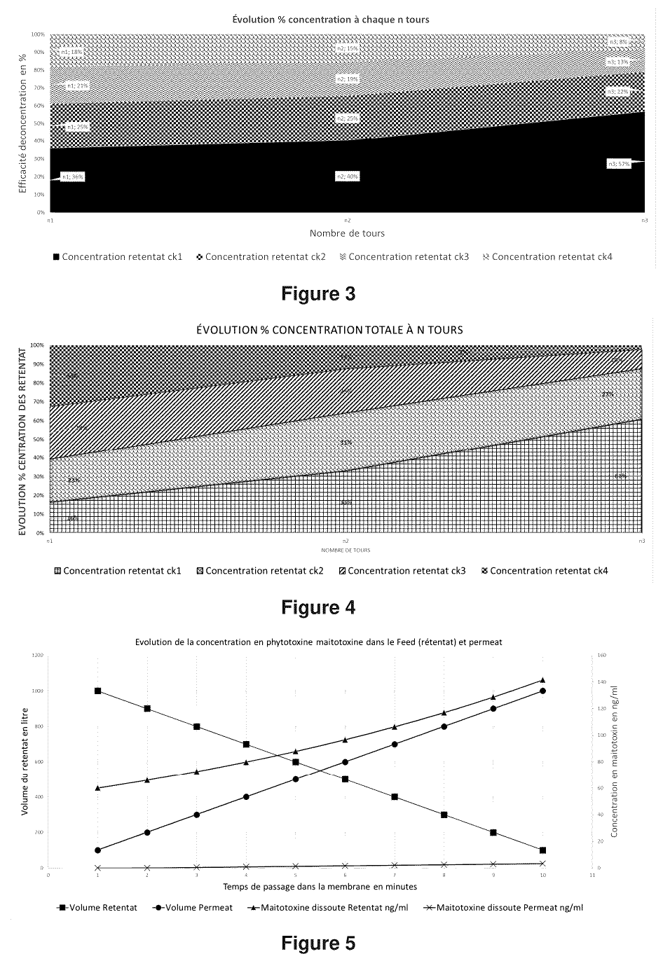

This method also ensures that the concentration of each batch of retentate increases with each pass (FIG. 3). The concentration factors denoted ck have the following values: ck1=5, ck2=2.5, ck3=1.6 and ck4=1.25.

The choice of membrane concentration factor will also influence the overall efficiency of the system. Depending on the type of batch you wish to produce, you will need to adapt the concentration factor of the membrane.

For example with the concentration factor of 1.6 (ck3) the evolution of the concentrations of the retentates remains constant.

But with a higher concentration factor such as 5 (ck1), the retentate concentration will only increase with each pass, allowing a greater variety of concentrations for recovery of multiple batches.

Yields are calculated with the following formula:

$$\% ( avec X = N ) \frac{{C}_{k [ λ ]}}{\sum Ntotale {C}_{k [ λ ]}}$$

### Example 2: Standardization of Different Batches of Biomass with Phytotoxin

A biomass of Gambierdiscus excentricus, known to produce a powerful toxin: maitotoxin, is cultivated in a 2.5 L photobioreactor, under artificial lighting with a wavelength between 400 and 460 nm and between 620 and 700 nm and an intensity of 350 μmol/m²/s, for 5 days until a dry matter of 1 g/l is obtained.

The biomass culture broth is then recovered and filtered through a 300 kiloDalton membrane to separate the biomass. The remaining permeate containing the phytotoxin then passes through a 1 kiloDalton filter, once. The 1000 liters of retentate will decrease and the phytotoxin will concentrate in the remaining retentate which will then be recovered and then analyzed by Liquid Chromatography coupled with Tandem Mass Spectrometry (LC-MS/MS). The evolution of the filtration (1000 Dalton filter) of the maitotoxin toxin (C164H258O68S2) with a size of 3380 Daltons via LC-MS/MS is monitored and presented in FIG. 5.

The retentate obtained after 10 minutes of filtration finally includes a greater concentration of maitotoxin than the initial retentate. It can then be distributed into different batches of biomass according to the maitotoxin concentrations in each batch to achieve an equal concentration in each batch.

1-10. (canceled)

11. A process for preparing a composition comprising a biomass of microorganisms, said preparation process comprising the addition of phytotoxins as an additive to said biomass, wherein the phytotoxins are obtained by a process comprising the steps of:
(a) membrane filtration of a biomass fermentation juice so as to retain the phytotoxins in the retentate, said biomass being obtained from a culture of phytotoxin-producing microorganisms,
(b) recovery of a retentate comprising said phytotoxins, and
(c) recovery of a first permeate depleted in phytotoxins, said permeate having a phytotoxin content lower than that of the fermentation juice,
followed if applicable by at least one additional cycle of membrane filtration of the retentate obtained in step (c) of the previous cycle with the recovery of at least a second retentate comprising said phytotoxins and at least a second permeate depleted in phytotoxins having a phytotoxin content lower than that of the first permeate.

12. The process according to claim 11, wherein the process for obtaining phytotoxins comprises m additional cycles of membrane filtration and the recovery of m+1 retentates comprising the phytotoxins and a m^{th} permeate depleted in phytotoxins, m being an integer of at minus 1.

13. The process according to claim 11, wherein the membrane filtration cycles are carried out on the same membrane or each membrane filtration cycle is carried out on a new membrane, the membranes of each filtration cycle being identical or different.

14. The process according to claim 11, wherein the number of cycles m+1 is determined as a function of:
the phytotoxin content of the fermentation juice,
the concentration factor of the filtration membrane, and
the final concentration of phytotoxins in the permeate recovered after the last cycle.

15. The process according to claim 14, the final concentration of phytotoxins in the permeate recovered after the last cycles as a function of the number of cycles m+1 is determined by the equation:
$${C}_{k [ λ ]} = {C}_{[ λ ]} * \frac{X}{X - 1} ( 1 - {X}^{R - 1} ) * {X}^{R}$$
in which λ is the number of cycles m+1, X is the membrane concentration factor and R is the membrane molecular rejection rate.

16. The process according to claim 12, the m+1 retentates comprising the phytotoxins recovered from the first membrane filtration cycle and m additional membrane filtration cycles are combined.

17. The process according to claim 11, the phytotoxins are molecules with a molecular weight of between 300 and 3500 Da.

18. The process according to claim 11, the phytotoxins belong to the families of microcystins, brevetoxins, saxitoxins, anatoxins a, okadaic acids, ciguatoxins, amphidinols, cylindrospermopsins, cyclic imines of ovatoxins, palytoxins and anabaenopeptins.

19. The process according to claim 11, the fermentation juice of step (a) is obtained by separation of a biomass of microorganisms and a fermentation must itself obtained by a process fermentation of said microorganisms in a culture medium comprising an aqueous medium and nutrients necessary for the growth of said microorganisms.

20. The process according to claim 12, the m^{th} permeate obtained after m additional cycles of membrane filtration is recycled in the culture medium of the microorganisms.
